(19) United States
(12) Patent Application Publication
(10) Pub. No.: US 2018/0271723 A1
(43) Pub. Date: Sep. 27, 2018

(54) HUMAN TRANSPORT DEVICE THAT FACILITATES DECONTAMINATION

(71) Applicant: ARC Products LLC, Des Peres, MO (US)

(72) Inventor: Clifford G. Adkins, Imperial, MO (US)

(21) Appl. No.: 15/922,314

(22) Filed: Mar. 15, 2018

Related U.S. Application Data
(60) Provisional application No. 62/474,338, filed on Mar. 21, 2017.

(51) Int. Cl. A61G 1/048; A61G 1/044

(52) U.S. Cl. CPC A61G 1/048; A61F 5/3769; A61G 1/044

(57) ABSTRACT

A transport device for use throughout a process of decontaminating a person exposed to contaminates is disclosed. The transport device includes a flexible sheet of material sized and shaped to support a person in a lying down position. The flexible sheet includes a base section and opposite side wall sections. The base section supports the person and the opposite side wall are configured to resiliently deflect upward along opposite sides of the person. The transport device includes a securing strap connected to the opposite side wall sections and configured to deflect the opposite side wall sections to secure the person on the flexible sheet. The transport device includes a drain in the base section. The drain permits liquid used in the process of decontamination to pass through and out of the flexible sheet. The drain includes a plurality of drain openings spaced apart from each other and distributed over the base section.

### CROSS-REFERENCE TO RELATED APPLICATIONS

This application claims priority to U.S. Provisional Patent Application Ser. No. 62/474,338, which is hereby incorporated by reference in its entirety.

### FIELD OF THE INVENTION

The present disclosure generally relates to human transport devices, and more particularly to human transport devices constructed to facilitate decontamination of non-ambulatory patients, such as may occur when hazardous substances are present at an accident site.

### BACKGROUND

When an accident, spill or emergency situation occurs with harmful and dangerous materials, the people involved can become contaminated. In order to treat these people, they must first be removed from the accident environment and decontaminated without contaminating others, such as first responders. Typically, decontamination involves moving the patient into a decontamination shower, were the patient is doused with water or other suitable decontaminating solution to wash away the contaminant. In situations where the patient is injured and or otherwise not ambulatory, it is necessary to provide a conveyance to move the patient and support the patient while decontamination occurs.

### SUMMARY

In one aspect of the present invention, a transport device for use throughout a process of decontaminating a person exposed to contaminates generally comprises a flexible sheet of material sized and shaped to underlie and support the person in a lying down position on the flexible sheet. The flexible sheet includes a base section and opposite side wall sections. The base section defines the bottom of the flexible sheet and supports the person on the base section. The opposite side wall sections extending along opposite sides of the base section are configured to resiliently deflect upward along opposite sides of the person. Strapping connected to the flexible sheet includes at least one securing strap connected to the opposite side wall sections and configured to extend across the person and deflect the opposite side wall sections to secure the person on the flexible sheet in the lying down position. A drain in the base section is configured to permit liquid used in the process of decontamination to pass through and out of the flexible sheet, the drain including a plurality of drain openings spaced apart from each other and distributed over the base section.

In another aspect, a method of decontaminating a person on a transport device generally comprises laying the person down on the transport device which has a main body comprising a flexible sheet of material sized and shaped to underlie and support a person lying down on the flexible sheet. The flexible sheet includes a base section defining the bottom of the flexible sheet and supporting the person thereon. The person on the transport device is decontaminated with a decontamination liquid. The decontamination liquid drains from the transport device through a plurality of drain openings in the base section. The person on the transport device is then carried from an area where decontamination has occurred into a treatment area.

### BRIEF DESCRIPTION OF THE DRAWINGS

FIG. 1 is a perspective of a transport device of the present disclosure;

FIG. 2 is top plan view of a flexible sheet of the transport device of FIG. 1, the flexible sheet being shown in a flat configuration and without strapping;

FIG. 3 is a top plan view of another embodiment of a flexible sheet of a transport device, the flexible sheet being shown in a flat configuration and without strapping; and

FIG. 4 a perspective of the transport device in a storage configuration.

Corresponding reference characters indicate corresponding parts throughout the drawings.

### DETAILED DESCRIPTION

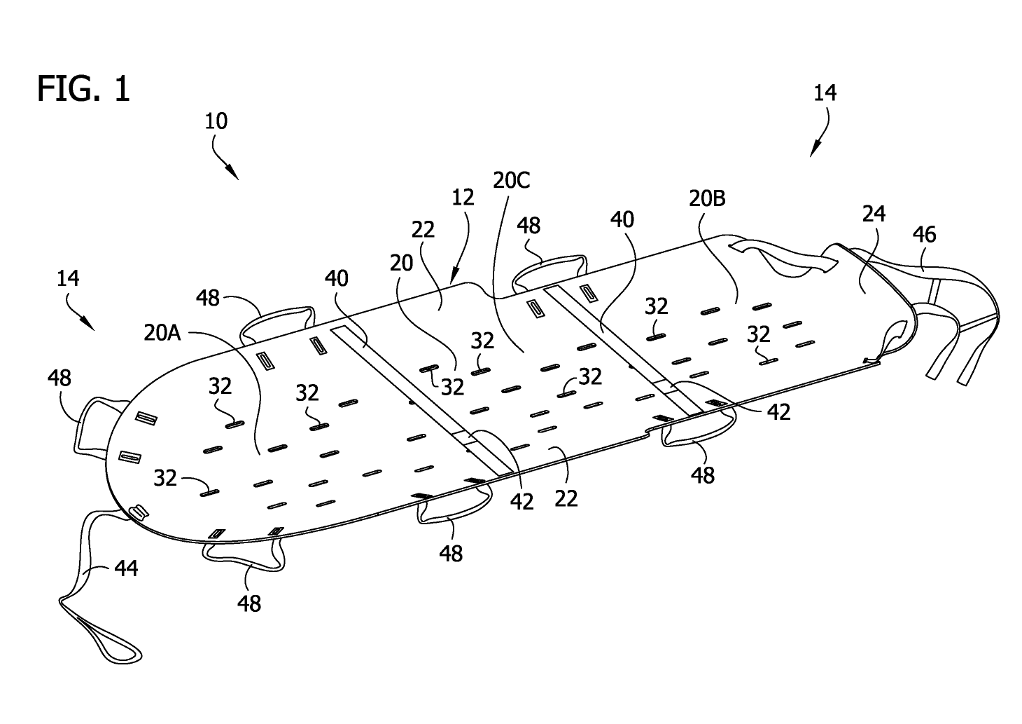

As shown in FIG. 1, a human transport device of the current disclosure is designated generally by the reference number 10. When there is an accident or spill that involves harmful chemicals, toxic solutions or powders, harmful transferable substances, or other contaminants, humans exposed to those conditions must be decontaminated. As will become apparent, the transport device 10 of the present disclosure can be used in various capacities throughout a process of decontaminating humans exposed to such contaminants.

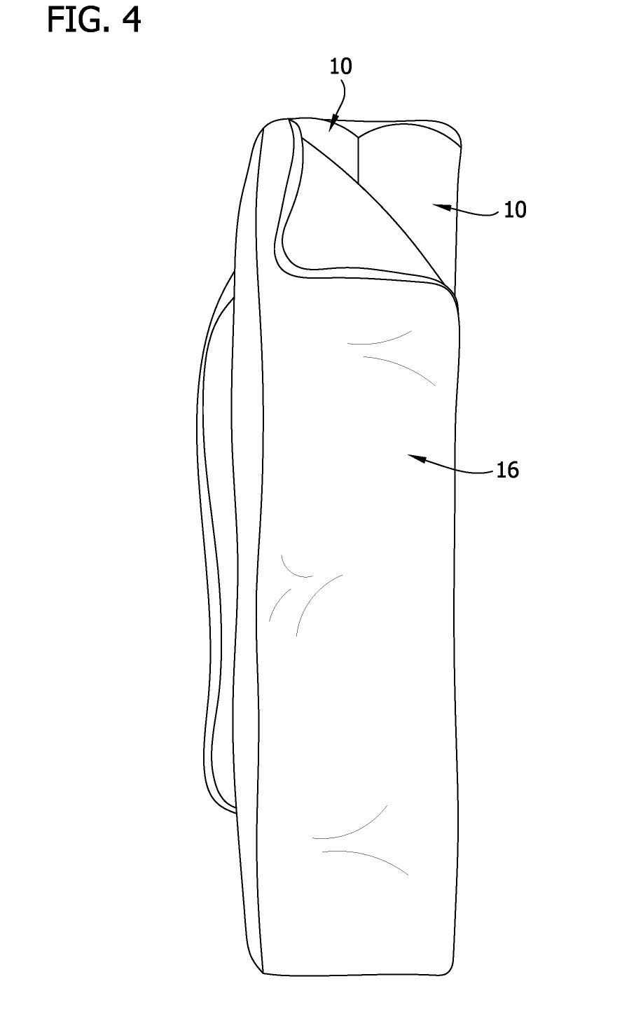

The transport device generally includes a main body 12 and strapping 14 for use in securing a human to the main body and/or moving the transport device. In the illustrated embodiment, the main body 12 comprises a sheet of flexible plastic, such as high density polyethylene, or another suitable material. In some embodiments, the material is selected to permit the transport device 10 to slide easily along the ground in all different types of terrain. The transport device 10 is configurable in a stowed configuration, such as shown in FIG. 4, in which the main body is rolled from head to toe into a compact cylinder-like shape and can be conveniently stored in a bag 16 (FIG. 4). In the illustrated embodiment, the bag 16 stores two transport devices 10. However, it is understood the bag 16 can be configured to store any number of transport devices 10. The transport device 10 can be referred to as a sled, for reasons which will become apparent.

When the transport device 10 is to be used, the main body 12 is unrolled, as shown in FIG. 1. The main body includes a base section or bed 20 sized for receiving a human thereon and wall sections 22, 24 configured to extend upwardly from the base section to assist in maintaining the human on the transport device 10 and specifically within the base section. It will be understood that the upstanding wall sections 22, 24 do not have to be present. In fact for a patient of large girth, the body may extend outside the base section 20 leaving very little or no portions of the wall sections 22 upstanding. In the illustrated embodiment, the base section 20 forms the bottom of the main body 12, and, as explained below, a bottom surface of the base section can function as a slide surface on which the main body is slidable over the ground or floor for dragging a person lying on the base section.

The wall sections 22, 24 include opposite side wall sections 22 and a foot wall section 24. The wall sections 22, 24 are resiliently bendable upward at the perimeter of the base section 20 for extending upwardly at the respective left and right sides and feet of a person lying on the base section. The arrangement is such that the person can be cocooned by the main body 12, rendering the flexible main body into a substantially rigid support on which the person can be dragged and/or carried.

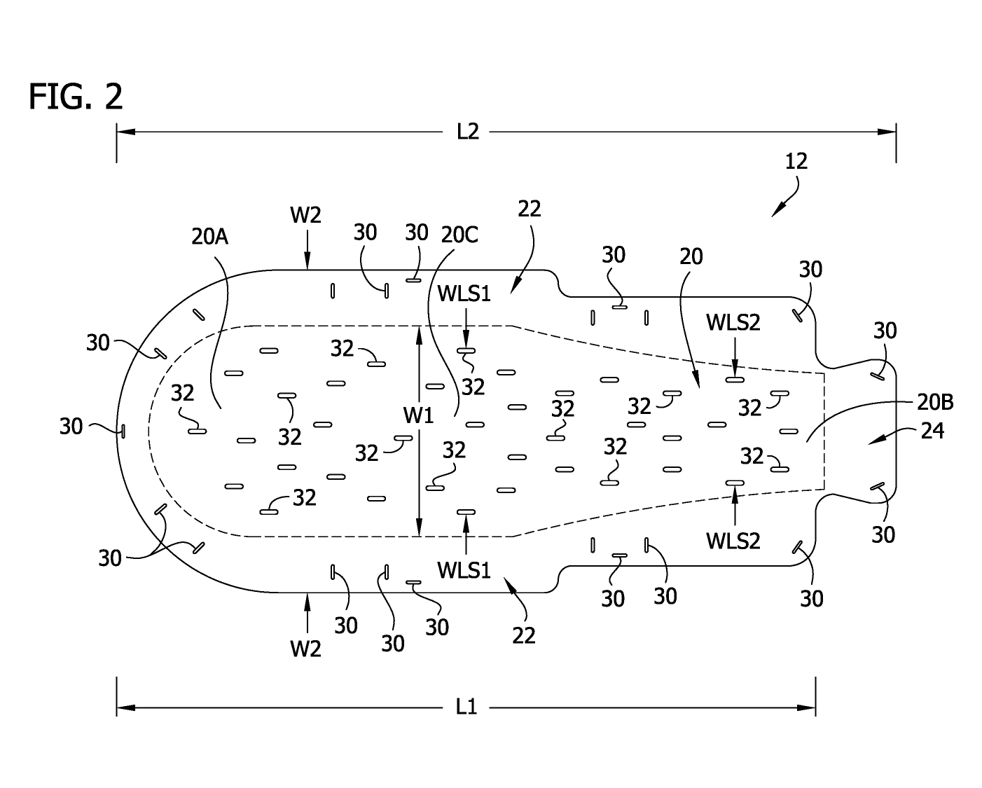

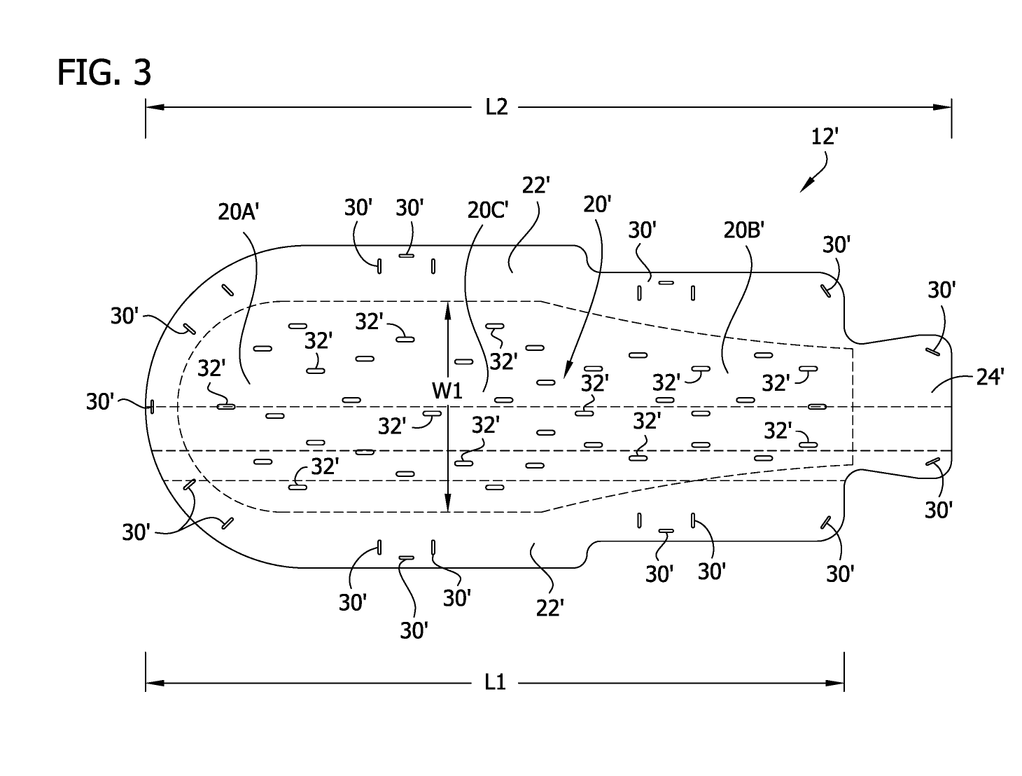

The main body 12 is shown in FIG. 2 without the strapping 14 and in a flat configuration. For example, the main body 12 can be cut or stamped out of a sheet of flexible plastic material. However it will be understood that other ways of forming the main body and other materials could be used, including without limitation rigid materials. The base section 20 and wall sections 22, 24 are separated by broken lines representing bend regions at which the main body 12 may be resiliently bent to extend the wall sections 22, 24 upwardly. The base section 20 has a length L1 rendering it suitable for supporting the human from head to toe. The base section has a head portion 20A, a leg portion 20B, and a length L1 extending from a head end of the head section to a tail end of the foot section. The length L1 can be in the range of 60-100 inches, such as between 65-85 inches. The base section 20 has opposite left and right sides and a width W1 extending therebetween transversely with respect to the length of the base section. Desirably, the width W1 of the base section 20 is in the range of 25-50 inches, such as between 30-40 inches, or about 36 inches. As shown in FIG. 2, in the illustrated embodiment, due to the foot wall section 24, the main body 12 has a length L2 greater than the length L1 of the base section 20, and, due to the side wall sections 22, the main body 12 has a width W2 greater than the width W1 of the base section. For example without limitation, certain example dimensions are indicated in FIG. 2. The dimensions of the transport device 10 can be other than described without departing from the scope of the present invention. For example and without limitation, in one embodiment, a bariatric transport device (not shown) may have different dimensions, such as a width of about 48 inches. Several holes 30 are formed in the main body 12 for receipt of the strapping 14. Several other holes 32 are formed in the base section 20 of the main body 12 to serve as the drain openings, as described below. An alternative larger main body 12′ having slightly different dimensions and shape is shown in FIG. 3.

Referring again to FIG. 1, the strapping 14 includes first and second securing straps 40 for securing the side wall sections 22 in their upwardly extending configurations and thus securing the person on the transport device. The securing straps 40 can include respective left and right strap sections connectable by a releasable quick-connect fastener 42. For example and without limitation, the fastener 42 could comprise as a snap buckle or seat belt type coupler. The strapping 14 also includes a tow member 44 at the head end of the main body 12 and a tow member 46 at the foot end of the main body. The tow member 46 at the foot end of the main body 12 is threaded through openings 30 in the foot wall section 24 and secured to the side wall sections 22. The foot tow member 46 assists in maintaining the foot wall section 24 extending upwardly from the base section. The foot tow member 46 includes a ladder-style strap assembly extending from the foot wall section 24 that can be used for towing and/or lifting the transport device. The strapping 14 also includes a plurality of handles 48 spaced around a periphery of the main body 12. In the illustrated embodiment, six handles 48 are provided and spaced along lengths of the side wall sections 22. The arrangement is such that two to eight people can assist in lifting the transport device 10, using the handles 48 and the head and foot tow members 44, 46. The flexible sheet of material forming the main body 12 and the connection of the handles 48 to the main body are such that an adult human being may be carried off the ground solely by persons gripping the handles without tearing or failing. It will be understood that the number of handles may be other than described without departing from the scope of the present invention. The flexible sheet forming the main body 12 is also constructed to slide easily over the ground, including paved and gravel surfaces.

The transport device 10 includes a plurality of drain openings 32 for permitting liquid to pass through the transport device. In particular, a plurality of drain openings 32 (collectively, a “drain”) are provided in the base section 20 of the main body 12. The drain openings 32 are spaced from one another and provided at the head portion 20A, foot portion 20B, and an torso portion 20C extending between the head and foot portions. In FIGS. 2 and 3, a closed dashed line indicates an outer boundary of all of the portions. The head portion 20A is the semi-circular portion at the left side of the drawings. The torso portion 20C lies generally where the dashed line defines a constant width section. The leg portion 20B begins where the dashed lines begin to taper toward one another at the right end of the torso portion 20C and continues to the transverse dashed line of the right side of the flexible sheet 20. The arrangement is such that the drain openings 32 permit liquid to drain through the base section 20 to under the transport device 10. The drain openings 32 are configured to prevent liquid from beading up on the edge of the opening (i.e. prevent the liquid's viscosity from keeping the liquid on the transport device 10). The drain openings 32 can have a slot (FIGS. 1-3) or an oval (not shown) shape to facilitate the movement of the liquid through the drain openings. The drain openings 32 can also have a rounded edge to facilitate the movement of liquid through the drain openings. Other shapes and configurations of the drain openings 32 are within the scope of the present invention. The drain openings 32 are staggered both widthwise and lengthwise of the main body 12 to reduce the chance that all openings will be blocked by the patient's body lying on the base section 20 of the main body. The drain openings 32 in the torso portion 20C have a maximum lateral separation MLS1 in a direction parallel to the width of the main body 12 and the drain openings 32 in the leg portion 20B have a maximum lateral separation MLS2. The maximum lateral separation MLS1 is greater than the maximum lateral separation MLS2. Without the drain openings 32, water or other liquid could pool in the transport device 10 or not readily drain off the base section 20.

A process of using the transport device 10 to decontaminate a human will be described with respect to three “zones” hereafter referred to hot, warm, and cold zones. The hot zone refers to a location of an accident. People must be extracted from this environment without contaminating others, including first responders. The transport device 10 can be used to move non-ambulatory people away from the location of the accident. For example, the transport device 10 can be used as a sled (e.g., by pulling one of the tow members 44, 46) and/or the transport device can be carried using one or more of the handles 48. In some cases, first responders will initiate some form of decontamination process by removing clothes and dousing the patient with water. The drain openings 32 of the transport device allow the contaminated water to flow through the transport device. The transport device can then be used as a stretcher to lift onto a gurney or waiting ambulance for transport.

The warm zone is typically a location for complete decontamination. Such locations are usually hospitals. National hospital protocols prohibit contaminated patients from entering the emergency room treatment area until they have had a decontamination shower. For ambulatory patients, most hospitals have built-in decontamination showers near emergency rooms or portable decontamination showers (e.g., in a parking lot). For non-ambulatory patients, roller-type beds are used, such as disclosed in U.S. Pat. No. 5,568,817. Such patients are showered and/or doused with water on the beds to wash away the contamination. The water is captured in one or more tanks below the roller beds. The transport device 10 can be used to allow the staff to move the patients from the ambulance to the washing roller beds by use of the handles 48. The drain openings 32 in the transport device 10 allow the water to rinse the patient and drain through the transport device, through the bed and then into the retention tanks. The patient can be rolled back and forth on the transport device 10 and showered to clean their entire body. The base section 20 of the transport device 10 provides a wide surface to log roll the patient during the process. After washing the person is complete, the transport device 10 acts as a stretcher to move the person to a waiting gurney. It will be understood that other ways of supporting the patient on the transport device 10 for showering may be used within the scope of the present invention.

The cold zone is where the patient is considered free of contamination and ready for further care. In the context of the hospital, the cold zone is usually the emergency room. After the washing process, the person is safe to move into the emergency treatment areas without fear of contaminating other patients, staff, or the facility itself.

Current procedures involve strapping patients onto back/spine boards and moving them though the decontamination shower process. Spine boards are rigid 7 foot poly/plastic boards with solid surfaces. The contaminated water and substances can pool on the boards. Common spine boards are only 16 to 18 inches wide and use woven fabric straps and buckles to hold patients to them. Hand holes around the perimeter of the boards create pinch zones for fingers and hands against a flat surface. These areas commonly tear decontamination gloves and suits. They are readily available but due to their limited width (16 to 18 inches) are extremely difficult and dangerous to log roll a patient on during decontamination of the patient's back areas. The boards have crevices, woven straps, grommets, etc. that retain water and contamination.

The transport device 10 desirably does not hold or trap contaminated water or the chemicals themselves. The main body 12 can be made of high density polyethylene, or another suitable material that is non-absorbent and can be cleaned with common decontamination wipes. Moreover, the strapping 14 of the transport device 10 can be formed of 100% non-absorbent coated webbing that is welded and sealed to prevent trapping or absorbing liquids. A coated metal tri glide can be used to hold the webbing to the main body 12 and can be removed or adjusted for complete cleaning. The straps 14 are attached to the sled body in a way that they do not create osmosis or water retention. Common decontamination wipes can be used for cleaning the straps. The handles 48 in the disclosed configuration allow for moving the patient on the transport device like a stretcher when the transport device is cocooned around the patient. In such a configuration, the transport device 10 provides a relatively rigid support for the patient. The straps 14 not only permit moving the patient from the contamination site, to pulling the stretcher from the ambulance, to moving the patient through the decontamination shower. The wide base section 20 (e.g., 36 inches) and the handle placement make for safer log rolling to wash all sides of the patient. The drain openings 32 of the transport device 10 permit quick shedding of contaminated water during the decontamination process. The several drain openings 32 spaced about the base section 20 of the transport device allows the water to drain quickly away from the patient, speeding the decontamination rinse process as well as reducing the amount of water needed to decontaminate the patient. The draining function allows for immediate reuse of the transport device 10 for another patient. The transport device 10 must be free of contaminants and contaminated water before the device can be reused for the next patient. Because the disclosed transport device 10 drains rapidly, provides a safer platform to log roll and rinse non-ambulatory patients, is extremely easy to clean, and is easy to store when not in use, the transport device provides an improved solution for the human decontamination process.

The transport device 10 may be carried by fire fighters and other first responders who will potentially need to rescue individuals from contamination sites. The transport device 10 enables the first responders to drag, carry, and pre-rinse a contaminated person near the point of contamination prior to transport to the nearest hospital. The transport device 10 may also be stored at hospitals for use in the decontamination washing process described above.

Although the transport device 10 is described herein as used for transporting humans and in a decontamination process, it will be understood that the transport device could be used in other scenarios. For example, the transport device 10 could be used in circumstances where it is desired to move non-human objects, or to move humans in conditions where rain, water from fire sprinklers, or other sources of water may make the transport device too heavy to move if the water is not quickly drained from the transport device. Persons having ordinary skill in the art will recognize the transport device could be used in many types of scenarios.

The transport device could have other constructions without departing from the scope of the present disclosure. For example, drain openings could be provided in additional or other locations, such as on the wall portions 22, 24.

In one alternative example, the main body may be formed of generally rigid material. The main body could include the base section formed of generally rigid material and have the drain openings as shown and described herein but lack the upstanding wall sections. The base section could have a width as described above permitting patients to be easily log rolled on the base section. The drain openings would permit liquid and contaminants to pass through the main body in washing procedures, and the generally rigid base section could serve as a back board for moving the patient. Handles such as straps shown herein could be provided around the periphery of the base section for lifting the transport device.

Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A transport device for use throughout a process of decontaminating a person exposed to contaminates, the transport device comprising:
a flexible sheet of material sized and shaped to underlie and support the person in a lying down position on the flexible sheet, the flexible sheet including a base section and opposite side wall sections, the base section defining the bottom of the flexible sheet and supports the person thereon, the opposite side wall sections extending along opposite sides of the base section and configured to resiliently deflect upward along opposite sides of the person;
strapping connected to the flexible sheet, the strapping including at least one securing strap connected to the opposite side wall sections and configured to extend across the person and deflect the opposite side wall sections to secure the person on the flexible sheet in the lying down position;
a drain in the base section, the drain configured to permit liquid used in the process of decontamination to pass through and out of the flexible sheet, the drain including a plurality of drain openings spaced apart from each other and distributed over the base section.

2. The transport device of claim 1 wherein the drain openings are each shaped to reduce beading up of liquid at an edge of the drain opening.

3. The transport device of claim 2, wherein the drain openings are elongate.

4. The transport device of claim 3 wherein the flexible sheet has a length and a longer dimension of the elongate drain openings are generally parallel to the length of the flexible sheet.

5. The transport device of claim 3 wherein each drain opening has an oval shape to prevent the liquid from beading up on an edge of each drain opening.

6. The transport device of claim 3 wherein each drain opening has a rounded edge

7. The transport device of claim 1 wherein the base section includes a head portion, a leg portion and a torso portion between the head and leg portions, the drain openings being positioned in the head, leg and torso portions.

8. The transport device of claim 7 wherein the drain openings in the torso portion have a maximum lateral separation in a direction widthwise of the flexible sheet and the drain openings in the leg portion have a maximum lateral separation in a direction widthwise of the flexible sheet, the maximum lateral separation of the drain openings in the torso portion being greater than the maximum lateral separation in the leg portion.

9. The transport device of claim 7 wherein the drain openings are staggered widthwise and lengthwise of the flexible sheet.

10. The transport device of claim 1, wherein the strapping is formed of 100% non-absorbent coated material to prevent the strapping from absorbing liquids.

11. The transport device of claim 1, wherein the at least one securing strap includes a releasable quick-connect fastener.

12. The transport device of claim 1, wherein the strapping is releasably connected to the flexible sheet with tri glides to limit the liquid retained by the flexible sheet.

13. The transport device of claim 12, wherein the tri glides are coated to protect the tri glides from the liquid used during decontamination.

14. The transport device of claim 1, wherein the flexible sheet further includes a foot wall section at a foot end of the base section, the foot wall section configured to resiliently deflect upward along the feet of the person.

15. The transport device of claim 14, wherein the strapping further includes first and second tow members, the first tow member connected to a head end of the base section and the second tow member connected to the foot end of the base section.

16. The transport device of claim 1, wherein the flexible sheet is nonabsorbent.

17. The transport device of claim 16, wherein the flexible sheet is a flexible sheet of plastic.

18. The transport device of claim 17, wherein the flexible sheet is configurable in a stowed configuration in which the flexible sheet is rolled into a compact cylinder-like shape to be stored in a bag.

19. The transport device of claim 1, wherein the strapping further includes a plurality of handles spaced around a periphery of the flexible sheet to facilitate the lifting of the transport device.

20. A method of decontaminating a person on a transport device, the method comprising:
laying the person down on the transport device, the transport device having a main body comprising a flexible sheet of material sized and shaped to underlie and support a person lying down on the flexible sheet, the flexible sheet including a base section defining the bottom of the flexible sheet and supporting the person thereon;
decontaminating the person on the transport device with a decontamination liquid, the decontamination liquid draining from the transport device through a plurality of drain openings in the base section;
carrying the person on the transport device from an area where decontamination has occurred into a treatment area.
